(19) United States
(12) Patent Application Publication
(10) Pub. No.: US 2007/0219247 A1
(43) Pub. Date: Sep. 20, 2007

(54) Medicament

(76) Inventor: Emiliangelo Ratti, Verona (IT)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/568,206

(22) PCT Filed: Apr. 21, 2005
(86) PCT No.: PCT/EP05/04401

§ 371 Date: Oct. 23, 2006

(30) Foreign Application Priority Data
Apr. 23, 2004 (GB) 0409098.1

(51) Int. Cl. A61K 31/454; A61P 25/22

(52) U.S. Cl. 514/326

(57) ABSTRACT

This invention provides the use of the compound [2-methoxy-5-(5-trifluoromethyl-tetrazol-1-yl-benzyl)]-([2S,3S]-2-phenyl-piperidin-3-yl)-amine, pharmaceutically acceptable salts or solvates thereof and pharmaceutical compositions containing them for the manufacture of a medicament for the treatment or prevention of Posttraumatic Stress Disorder (PTSD).

This invention relates to the use of the compound [2-methoxy-5-(5-trifluoromethyl-tetrazol-1-yl-benzyl)]-([2S,3S]-2-phenyl-piperidin-3-yl)-amine, pharmaceutically acceptable salts or solvates thereof and pharmaceutical compositions containing them in the treatment or prevention of Posttraumatic Stress Disorder (PTSD).

International patent application number WO95/08549 describes novel piperidine derivatives. One such compound described therein is [2-methoxy-5-(5-trifluoromethyl-tetrazol-1-yl-benzyl)]-(2-phenyl-piperidin-3-yl)-amine and it has the following chemical structure (I)

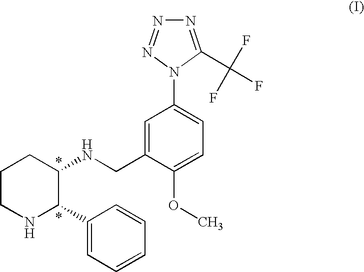

It will be appreciated by those skilled in the art that the compound of formula (I) contains two chiral centres (shown as * in formula (I)) and thus exists in the form of two pairs of optical isomers (i.e. enantiomers) and mixtures thereof including racemic mixtures.

For example, the compound of formula (I) may be either the cis isomer, as represented by figures (a) and (b), or the trans isomer, as represented by figures (c) and (d), or mixtures thereof.

All of the isomers of the compound of formula (I) represented by the figures (a) to (d) and mixtures thereof including racemic mixtures are included within the scope of the invention.

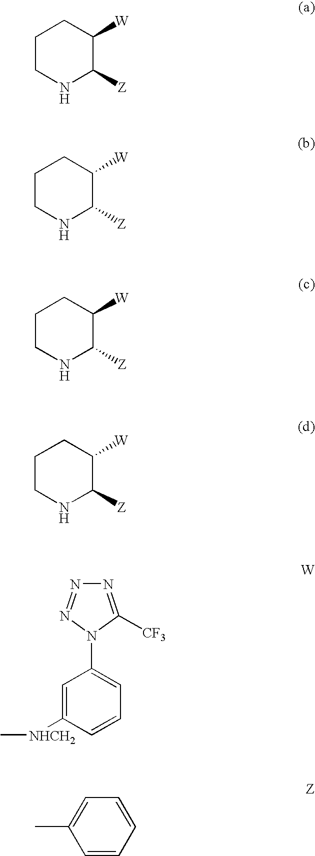

The compound of formula (I) may be in the form of the cis isomer (i.e. as represented by figures (a) and (b)), for example, the 2S, 3S isomer, [2-methoxy-5-(5-trifluoromethyl-tetrazol-1-yl-benzyl)]-([2S,3S]-2-phenyl-piperidin-3-yl)-amine, (i.e. as represented by figure (b)).

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include methanol, ethanol, acetic acid and water. When the solvent is water, the solvate may also be referred to as a hydrate.

It will be appreciated that for use in medicine the salts of formula (I) should be pharmaceutically acceptable. Suitable pharmaceutically acceptable salts will be apparent to those skilled in the art and include for example acid addition salts formed with pharmaceutically acceptable organic or inorganic acids for example, hydrochlorides, hydrobromides, sulphates, alkyl- or arylsulphonates (e.g. methanesulphonates or p-toluenesulphonates), phosphates, acetates, citrates, succinates, tartrates, fumarates and maleates.

Further examples of acid addition salts formed with inorganic acids are e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Examples of salts include the hydrochloride, maleate, tosylate or mesylate salts or pharmaceutically acceptable derivatives thereof. Other non-physiologically acceptable salts e.g. oxalates, may be used, for example in the isolation of the compound of formula (I) and are included within the scope of this invention. Also included within the scope of the invention are solvates and hydrates of the compound of formula (I). One such pharmaceutically acceptable salt of the compound of formula (I) for use according to the present invention is the dihydrochloride.

The compound of formula (I) may form acid addition salts with one or more equivalents of the acid. The present invention includes within its scope all possible stoichiometric and non-stoichiometric forms thereof.

Certain salts of the compound of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the salts of compounds represented by formula (I) as mixtures with isomers thereof in which one or more chiral centres are inverted. Likewise, it is understood that salts of compounds of formula (I) may exist in tautomeric forms other than that shown in the formula and these are also included within the scope of the present invention.

The term “pharmaceutically acceptable derivative” as used herein refers to any pharmaceutically acceptable derivative of a compound of the present invention, for example, an ester, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

The compound of formula (I) and salts and solvates thereof are described in WO95/08549 as potent and specific NK₁ receptor antagonists. The compound of formula (I) was initially evaluated for its use in the treatment and prevention of emesis.

The present invention relates to the further use of the compound of formula (I) or a pharmaceutically acceptable salt or solvate thereof is also useful in the treatment of Posttraumatic Stress Disorder (PTSD).

Posttraumatic Stress Disorder (PTSD) is a common chronic anxiety disorder that is often debilitating and follows exposure to an overwhelming traumatic event. Although disruptions in the hypothalamic-pituitary adrenal (HPA) Axis, noradrenergic, serotonergic systems have been proposed as neurobiological substrates in the development of PTSD, the exact underpinnings of the neurobiology of PTSD remain to be fully elucidated. PTSD is responsive to treatment with selective serotonin reuptake inhibitors, but response rates rarely exceed 60%, and even fewer patients (20%-30%) experience improvement that could be characterized as remission. Thus, there is a clear need to develop novel and improved therapeutics for PTSD.

Within the context of the present invention, the term ‘Posttraumatic Stress Disorder (PTSD)’ includes various disease states or specifiers, including acute, chronic and with delayed onset PSTD, which are classified in the Diagnostic and Statistical Manual of Mental Disorders, 4^{th} Edition, published by the American Psychiatric Association (DSM-IV), number 309.81. The various forms of the disorders mentioned therein are contemplated as part of the present invention.

We have found that the compound of formula (I) and its pharmaceutically salts and solvates thereof are useful in the treatment of PTSD.

In a first aspect thereof, the invention provides the use of the compound of formula (I) or a pharmaceutically acceptable salt or solvate thereof, for the manufacture of a medicament for the treatment of PTSD.

In a further aspect thereof, the invention provides the use of the compound of formula (I) or a pharmaceutically acceptable salt or solvate thereof, for the treatment of PTSD.

In a yet further aspect, the invention provides a method of treatment of PTSD which comprises administering to a human in need thereof an effective amount of the compound of formula (I) or a pharmaceutically acceptable salt or solvate thereof.

In a yet further aspect thereof, the present invention provides a pharmaceutical composition which comprises the compound of formula (I) or a pharmaceutically acceptable salt or solvate thereof for the treatment or prevention of PTSD.

Pharmaceutical compositions for use in accordance with the present invention may be formulated in conventional manner using one or more pharmaceutically acceptable carriers or excipients.

It will be appreciated that reference to treatment is intended to include prophylaxis as well as the alleviation of established symptoms.

Thus, the compound of formula (I) and its pharmaceutically acceptable salts and solvates may be formulated for oral, buccal, parenteral, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compound of the invention and its pharmaceutically acceptable salts and solvates may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compound of the invention and its pharmaceutically acceptable salts and solvates may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of the invention and its pharmaceutically acceptable salts and solvates may also be formulated in rectal compositions such as suppositories or retention enemas, e.g. containing conventional suppository bases such as cocoa butter or other glycerides.

The compound of the invention and its pharmaceutically acceptable salts and solvates may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of the invention and its pharmaceutically acceptable salts and solvates may be formulated as solutions for administration via a suitable metered or unitary dose device or alternatively as a powder mix with a suitable carrier for administration using a suitable delivery device.

A proposed dose of the compound of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus, for parenteral administration a daily dose will typically be in the range of 1 to about 100 mg, such as 1 to 80 mg per day. For oral administration a daily dose will typically be within the range 1 to 100 mg e.g. 10 to 50 mg.

The compound of formula (I) and its pharmaceutical salts and solvates thereof may be prepared by the process described in international patent application no. WO95/08549, which is incorporated herein by reference.

It will be appreciated by those skilled in the art that the compound of formula (I) or pharmaceutically acceptable salts or solvates thereof according to the invention may advantageously be used in conjunction with one or more other therapeutic agents, for instance, 5-HT reuptake inhibitors (such as escitalopram, escitalopram oxalate, sertraline, fluoxetine or paroxetine), mixed 5HT and NE reuptake inhibitors (such as venlafaxine or duloxetine), NE reuptake inhibitors (such as reboxetine), GABA receptor agonists (such as topiramate), 5-HT2A antagonists (such as risperidone), dopamine reuptake inhibitors (such as bupropion), GABA uptake inhibitors (such as tiagabine), alpha 2 adrenoreceptor antagonists (such as mirtazapine), COX-2 inhibitors (such as celecoxib or rofecoxib) and atypical antipsycotics (such as aripiprazole).

Pharmacological Activity

The invention may be illustrated by suitable patient studies. The following example of a suitable patient study is for illustrative purposes and is not intended to limit the scope of the invention in any way. The study is a 8-week randomized double-blind, placebo-controlled parallel study of the effect of [2-methoxy-5-(5-trifluoromethyl-tetrazol-1-yl-benzyl)]-(2-phenyl-piperidin-3-yl)-amine dihydrochloride in approximately 52 patients meeting the criteria for PTSD, according to DSM-IV and confirmed by the Structured Clinical Interview for the Diagnostic Manual of Mental Disorders, 4^{th} Ed., Patient Version (SCIP-P) (First et al.). The acute efficacy of the NK1 antagonist [2-methoxy-5-(5-trifluoromethyl-tetrazol-1-yl-benzyl)]-(2-phenyl-piperidin-3-yl)-amine dihydrochloride (GR205171) in improving overall PTSD symptomatology will be measured by the mean change in the Clinician-Administered PTSD scale (CAPS) total score from baseline score to the score at the end of 8 weeks of therapy.

Study Phases

After patients' consent, the patients will undergo several procedures. This will include: 1) psychiatric interview by a research psychiatrist; 2) structured interview (the SCID); 3) Clinician Administered PTSD Scale (CAPS); 4) collection of blood samples for routine medical tests, thyroid function tests, and urine sample [urine analysis and toxicological screen]; 6) neurological and physical examination. The SCID and rating scales will be performed by a research psychiatrist, a masters-level research social worker, clinical graduate students in psychology, or a research R.N. with specialized training in diagnostic assessment.

The design consists of a 14-day drug washout and two study periods:

Drug washout phase (Day -28 to -14): Subjects will be tapered off psychotrpic medications over 7-14 days, generally corresponding to over five ½ lives [fluoxetine within 6 weeks is an exclusion]. Sedative/hypnotic drugs are not permitted during the study.

Study period I (Day -14 to -1): After completing the washout phase, subjects will receive single-blind placebo lead-in during a period of 14 days prior to randomisation to exclude subjects who have a greater than 25% improvement in CAPS between Visit 1 and 2. During this period of time, subjects will have potential surrogate marker testing: neuroimaging, psychophysiological and neuroendocrine tests (Day -7 through day -1).

Study period 11 (Day 0 to 56): This is an 8-week double-blind therapy period. Patients will be assessed weekly from Visit 2 through Visit 10. Subjects will be randomized at a ratio of 1:1 to receive either NK₁ antagonist [2-methoxy-5-(5-trifluoromethyl-tetrazol-1-yl-benzyl)]-(2-phenyl-piperidin-3-yl)-amine dihydrochloride GR205171 or placebo. Therapy will be initiated at a dose of 5 mg/day of NK1 antagonist or matching placebo. No other psychotropic medication will be permitted during Study Period II. At the end of this period, neuroimaging and neuroendocrine tests will be repeated. Patients are outpatients during the duration of the study. The surrogate marker testing is repeated during days 49 to 56.

Rating Scales

The primary intent of this study is to compare the efficacy of NK-1 antagonist relative to placebo in the treatment of overall anxiety symptomatology of subjects who have PTSD. This will be measured by comparing reductions from baseline scores on the CAPS total score after up to 8-weeks of double-blind therapy to the original score before the start of therapy. To test treatment efficacy, random effects regression will be used to contrast study groups for continuous measures. Pearson's chi-squared test or Fisher's exact test will be used for analysis of proportions. All hypotheses will be tested at a two-sided a level of 0.05. Additional analyses will be performed for origin, gender, age, dose level and certain illness characteristics (if there are at least 10 subjects in each treatment group). All subgroup analyses will be considered secondary analyses.

Clinician-Administered PTSD Scale (CAPS)

The CAPS is a structured clinical interview designed to assess the essential features of PTSD as defined by the DSM-IV. The CAPS can be used to provide categorical ratings of diagnostic status as well as a quantitative index of symptom severity. Both frequency and intensity scores are derived for each individual symptom. Frequency ratings are made on a 5-point scale (0=never; 4=daily or all the time). Symptom intensity ratings are made on a 5-point scale (0=none or no problem with symptoms; 4=extreme, incapacitating). The CAPS total severity score is based on the subject's response to the 17 items that assess the frequency and intensity of current PTSD symptoms. The total severity score ranges from 0-136. Higher scores reflect a greater degree of symptoms severity.

Subscales of the CAPS will be utilized to assess specific symptom clusters. Subscales of the CAPS assess both the frequency and intensity of the specific symptom clusters (sum of items 6 through 12), hyperarousal (sum of items 13 through 17), and dissociative (sum of items 28 through 30).

Structured Clinical Interview for DSM-IV-TR Axis I Disorders Patient Version (SCID I/P)

The SCID I/P (First et al 2001) is a semi-structured interview. It is administered by a clinician to diagnose psychiatric illness. It provides probe questions as well as follow-up questions to be asked by the clinician to assist in diagnosis. It includes an overview to obtain information about demographics, work, chief complaint, history of present illness, past history, treatment history, and current functioning. The main body of SCID includes 9 modules that are designed to diagnose 51 mental illnesses in all. The modules of the research version can be tailored to needs, purpose, and goals of the investigation. It includes sections on current as well as past psychiatric disorders.

All publications, including but not limited to patents and patent applications, cited in this specification are herein incorporated by reference as if each individual publication were specifically and individually indicated to be incorporated by reference herein as though fully set forth.

1. (canceled)

2. A method of treatment of Posttraumatic Stress Disorder (PTSD) which comprises administering to a human in need thereof an effective amount of the compound of formula (I) 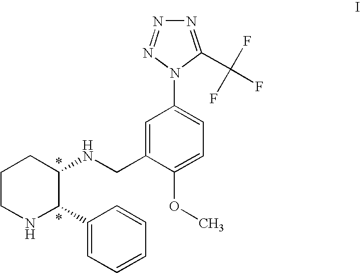
wherein * represents a chiral centre, or a pharmaceutically acceptable salt or solvate thereof.

3-4. (canceled)

5. The method according to claim 2 wherein the compound is [2-methoxy-5-(5-trifluoromethyl-tetrazol-1-yl-benzyl)]-([2S,3S]-2-phenyl-piperidin-3-yl)-amine or a pharmaceutically acceptable salt or solvate thereof.

6-7. (canceled)

8. The method according to claim 2 wherein the pharmaceutically acceptable salt is the dichloride.

9. (canceled)

10. The method according to claim 5 wherein the pharmaceutically acceptable salt is the dichloride.
